(19) United States
(12) Patent Application Publication
(10) Pub. No.: US 2017/0258879 A1
(43) Pub. Date: Sep. 14, 2017

(54) METHOD OF TREATING SEASONAL AFFECTIVE DISORDER

(71) Applicants: Eric Finzi, (US); Norman E. Rosenthal, (US)

(72) Inventors: Eric Finzi, Washington, DC (US); Norman E. Rosenthal, Rockville, MD (US)

(21) Appl. No.: 15/455,032

(22) Filed: Mar. 9, 2017

Related U.S. Application Data
(60) Provisional application No. 62/308,149, filed on Mar. 14, 2016.

(51) Int. Cl. A61K 38/48; A61K 45/06

(52) U.S. Cl. CPC A61K 38/4893; C12Y 304/24069; A61K 45/06

(57) ABSTRACT

Methods are provided for treating or preventing seasonal affective disorder caused by the onset of a specific season in a subject. The method include administering a therapeutically effective amount of a neurotoxin to a facial muscle to cause paralysis of the facial muscle, thereby affecting the ability of the subject to frown and thus treating or preventing the seasonal affective disorder prior to the onset of the season.

### PRIORITY CLAIM

This claims the benefit of U.S. Provisional Application No. 62/308,149, filed Mar. 14, 2016, which is incorporated by reference herein.

### FIELD

This relates to the field of psychiatric disorders, and in particular to a method for preventing, or delaying the onset of symptoms of, seasonal affective disorder, specifically seasonal depression, that utilize a neurotoxin such as botulinum toxin A.

### BACKGROUND

Seasonal affective disorder (SAD), also called seasonal depression, is a specific type of depression that occurs during the same season each year. SAD is diagnosed if a patient feels depressed during the last two specific seasons, such as the last two winters, but feels much better in other seasons, such as spring and summer. Anyone can get SAD, but it's more common in women, people who live far from the equator, and people who are between the ages of 15 and 55. There is a family association with this disorder, so that a person with a close relative with SAD is more susceptible.

It has been proposed that SAD that occurs in fall and winter (known as winter SAD) is caused by a lack of sunlight, or the shortening length of the day. It has also been proposed that SAD is caused by differences in serotonin levels, which affect mood.

The symptoms of SAD include feeling depressed and/or anxious, loss of interest in activities during a specific season, and changes in eating habits, weight, sleep and concentration during a specific season. Generally, the symptoms appear and disappear at about the same time every year.

A constant sense of hopelessness and despair is a sign that a subject can have major depression, also known as clinical depression. With major depression, it can be difficult to work, study, sleep, eat, and enjoy friends and activities. vSome people have clinical depression only once in their life, while others have it several times in a lifetime.v Major depression seems to occur from one generation to the next in some families, but may affect people with no family history of the illness. Most people with SAD start to have symptoms in September or October and feel better by April or May. Existing treatments for SAD are not always fully effective. Therefore, a need remains for the treatment of SAD. There is also a need for agents for the prevention of SAD, that can be administered prior to when the illness is likely to occur.

### SUMMARY

Methods are disclosed herein for treating seasonal affective disorder. The subject can have a disorder including or consisting of the seasonal affective disorder (SAD). The methods include administering a therapeutically effective amount of a neurotoxin to the corrugator supercilli and/or the procerus muscle of the subject to cause paralysis, thereby treating the SAD. The method can include administering the neurotoxin to the subject prior to the time (season) of the onset of the seasonal affective disorder. Surprisingly, it was demonstrated that, onset of the SAD can be delayed, such as to an extent that the subject does not develop SAD during a particular season. Thus, the disclosed treatment can be administered in specific seasons, and not utilized at other times of the year. SAD can be prevented by initiating treatment with Botulinum toxin A before the onset of symptoms, and it can be administered at intervals throughout the season of risk, thereby preventing the development of symptoms, and can be discontinued once the season of risk has passed.

In some embodiments, methods are provided for treating or preventing seasonal affective disorder caused by the onset of a specific season in a subject. The method include administering a therapeutically effective amount of a neurotoxin to a facial muscle to cause paralysis of the facial muscle, thereby affecting the ability of the subject to frown and thus treating or preventing the seasonal affective disorder prior to the onset of the season.

In additional embodiments, methods are provided for treating or preventing seasonal affective disorder in a subject, comprising administering a therapeutically effective amount of a Botulinum toxin A to a glabella of the subject to cause paralysis of a facial muscle in the glabella, thereby affecting the ability of the subject to scowl or appear sad, and thus treating or preventing seasonal affective disorder in the subject. The seasonal affective disorder is caused by onset of a specific season, and the Botulinum toxin A is administered about two to about six weeks prior to the onset of the specific season.

In some embodiments, SAD is prevented by initiating treatment with Botulinum toxin A before the onset of symptoms, and administering Botulinum toxin A at intervals throughout the season of risk, thereby preventing the development of symptoms. In further embodiments, Botulinum toxin A is discontinued once the season of risk is passed.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram of a frontal view of the musculature of the human face and neck. Injection sites for a neurotoxin, such as Botullinum toxin (BTX) into the glabella, which includes the procerus and the corrugator supercilii muscles, are shown.

FIGS. 2A-2B show Botulinum toxin dose used to treat the frown in a typical woman (FIG. 2A) and man (FIG. 2B). The landmarks for the injection are as follows (see also Carruthers et al., Derm. Surg. 24:1189-1194, 1998). The injection of the procerus is below a line joining the medial end of both eyebrows. The landmark for the next injections is a line vertically above the inner canthus and the superior margin of the orbit. Botulinum toxin is injected just superior to the first injection point. Next an injection is made 1 centimeter (cm) medial and one-half cm superior to the second two injections line. The numbers refer to the number of BTX Units injected in the area shown.

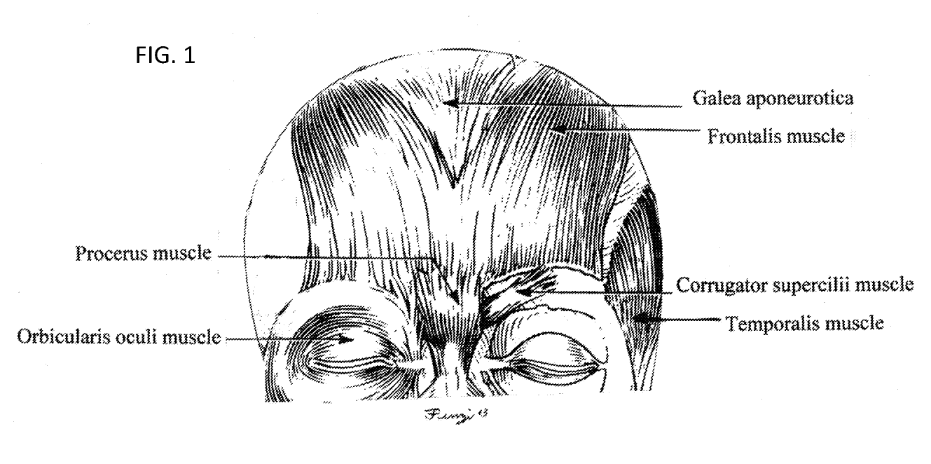

### DETAILED DESCRIPTION

Methods are disclosed herein for treating seasonal affective disorder. The subject can have a disorder including or consisting of the seasonal affective disorder (SAD). The methods include administering a therapeutically effective amount of a neurotoxin to the corrugator supercilli and/or the procerus muscle of the subject to cause paralysis, thereby treating the SAD. The method can include administering the neurotoxin to the subject prior to the season of onset of the seasonal affective disorder, thereby preventing onset of symptoms until the season of risk has passed. In some embodiments, the neurotoxin is administered at intervals throughout the season of risk and/or administration of the neurotoxin is discontinued once the season of risk is passed.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Absent an underlying physical disorder: A phrase used to describe a muscular disorder (for example, torticollis) or cosmetic issue (for example, wrinkles) that is already known to be reduced or prevented by treatment with botulinum toxin that is not present in a subject. For example, the method includes selecting a subject that does not have a muscular disorder or condition, such as a subject that does not have spasms, cramping, tightness of muscles caused by medical problems, or torticollis. A physical condition can be someone that has need and/or interest in cosmetic application of Botulinum toxin, such as to decrease wrinkles. For example, the method includes selecting a subject that does not have an underlying cosmetic issue (such as wrinkles) and/or is not selected for treatment for this underlying cosmetic disorder.

Ameliorating or ameliorate: Any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation. For example, a clinical guide to monitor the effective amelioration of a psychiatric disorder, such as depression, is found in the Structured Clinical Interview for DSM-IV Axis I mood disorders (“SCID-P”) (see fourth edition of Diagnostic and Statistical Manual of Mental Disorders (1994) Task Force on DSM-IV, American Psychiatric Association (“DSM-IV”); Kaplan, Ed. (1995); Comprehensive Textbook of Psychiatry/VI, vol. 1, sixth ed., pp 621-627, Williams & Wilkins, Baltimore, Md.).

Administration: To provide or give a subject an agent by any effective route. Exemplary routes of administration include, but are not limited to, oral; injection, continuous or intermittent infusion (such as subcutaneous, intramuscular, intradermal, intrathecal, epidural, intracranial, intraperitoneal, and intravenous); sublingual; rectal; transdermal; intranasal; vaginal; and inhalation routes.

Anti-Depressant Medications: A pharmaceutical agent that can be administered as a treatment for depression but are also administered for other conditions such as anxiety disorders, obsessive compulsive disorder, chronic pain, attention deficit disorder, substance abuse and sleep disorders. Anti-depressant medications include synthesized chemical compounds as well as naturally occurring or herbal remedies such as St. John's Wort. Generally, these medications are administered orally, but they may also be administered in any form of use to a medical practitioner. Examples of antidepressant medications include tricyclic antidepressants, which generally affect the two chemical neurotransmitters, norepinephrine and serotonin. Tricyclics include imipramine, amitriptyline, nortriptyline, and desipramine. Monoamine oxidase inhibitors (MAOIs) are also used as antidepressants. MAOIs approved for the treatment of depression include phenelzine (NARDIL®), tranylcypromine (PARNATE®), and isocarboxazid (MARPLAN®). Medications that primarily affect the neurotransmitter serotonin, termed selective serotonin reuptake inhibitors (SSRIs), are also used as antidepressants. These include escitalopram HBr (LEXAPRO®), fluoxetine (PROZAC®), sertraline (ZOLOFT®), fluvoxamine (LUVOX®), paroxetine (PAXIL®), and citalopram (CELEXA®). Additional medication of use affect both norepinephrine and serotonin, for example venlafaxine (EFFEXOR®) and nefazadone (SERZONE®), or agents such as phenelzine (NARDIL®), tranylcypromine (PARNATE®), mirtazepine (REMERON®), nefazodone (SERZONE®), triazolopyridine (TRAZODONE®), and bupropion (WELLBUTRIN®).

Anxiety disorder: Anxiety disorders include disorders that share features of excessive fear and anxiety related behavioral disturbances. Fear is the emotional response to real or perceived imminent threat, whereas anxiety is anticipation of future threat. These two states overlap but differ. Fear is more often associated with surges of autonomic arousal necessary for fight or flight, thoughts of immediate danger, and escape behaviors, while anxiety is more often associated with muscle tension and vigilance in preparation for future danger and cautious or avoidant behaviors. Sometimes the level of fear or anxiety is reduced by pervasive avoidance behaviors. Panic attacks feature prominently within the anxiety disorders as a particular type of fear response. Panic attacks are not limited to anxiety disorders but rather can be seen in other mental disorders as well.

The anxiety disorders differ from one another in the types of objects or situations that induce fear, anxiety, or avoidance behavior, and the associated cognitive ideation. Thus, while the anxiety disorders tend to be highly comorbid with each other, they can be differentiated by close examination of the types of situations that are feared or avoided and the content of the associated thoughts or beliefs. Anxiety disorders include generalized anxiety disorder, phobias, panic disorder, agoraphobia, social anxiety disorder, obsessive-compulsive disorder, separation anxiety, and situation anxiety.

Benzodiazepine: A group of psychoactive medications whose core chemical structure is the fusion of a benzene ring and a diazepine ring, that are used for treating panic disorder, insomnia, and generalized anxiety disorder, amongst other indications. Benzodiazepines include, but are not limited to, 2-keto compounds (such as chlordiazepoxide, clorazepae, diazepam, flurazepam, halazepam, and prazepam), 3-hydroxy compounds (such as lorazepam, lormetazepam, oxazepam, and temazepam), 7-nitro compounds (such as clonazepam, flunitrazepam, nimetazepam, and nitrazepam), Triazolo compounds (such as adinazolam, alprazolam, estazolam, triazolam, and imidazo compounds (such as climazolam, loprazolam, and midazolam).

Blood Brain Barrier: A separation of circulating blood from the brain extracellular fluid (BECF) in the central nervous system (CNS). It occurs along all capillaries and is composed of tight junctions around the capillaries that do not exist in the general circulation. This barrier also includes a thick basement membrane and astrocytic endfeet. This barrier restricts the diffusion of microscopic objects (such as bacteria) and other molecules, such as toxins, large molecules, and/or hydrophilic molecules into the cerebrospinal fluid (CSF), while allowing the diffusion of small hydrophobic molecules (O₂, CO₂, hormones). Cells of the barrier actively transport metabolic products such as glucose across the barrier with specific proteins.

Botulinum toxin: A toxin produced by the bacterium Clostridium botulinum, but which may either be obtained from a natural source or made by synthetic means. Seven immunologically distinct Botulinum neurotoxins have been characterized, these being respectively Botulinum neurotoxin serotypes A, B, C₁, D, E, F and G, each of which is distinguished by neutralization with type-specific antibodies. The different serotypes of Botulinum toxin vary in the animal species that they affect and in the severity and duration of the paralysis they evoke. For example, it has been determined that Botulinum toxin type A is 500 times more potent, as measured by the rate of paralysis produced in the rat, than is Botulinum toxin type B. Additionally, Botulinum toxin type B has been determined to be non-toxic in primates at a dose of 480 U/kg which is about 12 times the primate LD₅₀ for Botulinum toxin type A (Moyer et al., “Botulinum Toxin Type B: Experimental and Clinical Experience,” in Therapy With Botulinum Toxin, Marcel Dekker, Inc. Jankovic et al. (eds.), pgs. 71-85, 1994).

Depressive Disorder: A mood disorder characterized by a predominantly sad or depressed mood, typically but not always of two or more weeks' duration. A depressive disorder also has other signs or symptoms accompanying a sad or depressed mood, including one or more of: decreased energy, appetite changes, weight gain or loss, insomnia or hypersomnia, recurrent thoughts or death, thoughts of suicide, loss of interest in usual activities, slowed thinking or cognitive speed, increased speech latency, decreased volume of speech, excessive or inappropriate guilt, diminished concentration, feeling sluggish, and slower than normal motor activity (such as gross motor, fine motor, speech). Depressive disorders can be accompanied by perceptual disturbances. Depressive disorders can be caused by a medical disorder (e.g., endocrine disorders, lupus), medication side-effect (e.g., interferon), substance use disorder, neurologic disorder (e.g., seizure disorder, traumatic brain injury), or have no clear cause.

Depression: A mental state of depressed mood characterized by feelings of sadness, despair and discouragement. Depression includes feelings of sadness considered to be normal (mild depression), dysthymia, and major depression. Depression can resemble the grief and mourning that follows bereavement, and there are often feelings of low self esteem, guilt and self reproach, withdrawal from interpersonal contact and somatic symptoms such as alterations in eating habits and sleep disturbances.

Frown: To furrow the brow to show unhappiness or displeasure. This action uses the orbicularis oculi, the frontalis muscle and/or the corrugator supercilii muscle. This action can also include the use of the procerus muscle.

Glabella: The area, including the skin and muscles, between the eyebrows and above the nose. The underlying bone is slightly depressed, and joins the two superciliary ridges. The glabella is a cephalometric landmark just superior to the nasion.

Hallucination: Altered, misperceived, or incorrect sensory experiences. See “perceptual disturbances” below for additional information.

Light Therapy: Exposure to bright light, wherein individual sits in front of a source of bright light unit, usually balanced spectrum fluorescent tubes. The most common device used for bright light therapy is a fluorescent light box which produces a light intensity of 2,500 to 10,000 lux at a comfortable distance (such as about 1 to about 2 inches from the subject). The light can be of any spectrum, but usually includes visible light, and often does not include UV light.

Mood: A person's subjective report on their emotional perspective on self, situation, future, or past. While mood can fluctuate from states such as “happy,” “sad,” “angry,” or “pleased” within a day, a prolonged state of sad or depressed mood is a defining characteristic of a depressive illness.

Mood disorder: A medical, neurologic, or psychiatric disorder with the primary sign or symptom as an alteration in mood. Mood disorders are usually classified as depressive (e.g., principal mood symptom is a sustained sad or depressed mood) or manic (e.g., principal mood symptom is a sustained expansive, elevated, or irritable mood). Symptoms or signs beyond the mood state proper may be required to diagnose a mood disorder.

Nightmare: A frightening dream that causes the interruption of sleep. Repeated instances of nightmares can lead to a specific sleep disorder diagnosis of Nightmare Disorder. Nightmares are also commonly observed as a symptom in PTSD and other anxiety conditions.

Obsessive Compulsive Disorder: A pervasive pattern of preoccupation with orderliness, perfectionism, and mental interpersonal control, at the expense of flexibility, openness, and efficiency, beginning by early adulthood and present in a variety of contexts, as indicated by four (or more) of the following:

1. Is preoccupied with details, rules, lists, order, organization, or schedules to the extent that the major point of the activity is lost.

2. Shows perfectionism that interferes with task completion.

3. Is excessively devoted to work and productivity to the exclusion of leisure activities and friendships (not accounted for by obvious economic necessity).

4. Is overconscientious, scrupulous, and inflexible about matters of morality, ethics or values.

5. Is unable to discard worn-out or worthless objects even when they have no sentimental value.

6. Is reluctant to delegate tasks or to work with others unless they submit to exactly his or her way of doing things.

7. Adopts a miserly spending style towards both self and others; money is viewed as something to be hoarded for future catastrophes.

8. Shows rigidity and stubbornness.

Thus, obsessive compulsive disorder can be characterized by at least 4, 5, 6, 7 or all 8 of these characteristics.

Panic Attack: An abrupt surge of intense fear or intense discomfort, that can occur from a calm state or an anxious state, that reaches a peak within minutes, and during which four (or more) of the following symptoms occur:

1. Palpitations, pounding heart, or accelerated heart rate.

2. Sweating.

3. Trembling or shaking.

4. Sensations of shortness of breath or smothering.

5. Feelings of choking.

6. Chest pain or discomfort.

7. Nausea or abdominal distress.

8. Feeling dizzy, unsteady, light-headed, or faint.

9. Chills or heat sensations.

10. Paresthesias (numbness or tingling sensations).

11. Derealization (feelings of unreality) or depersonalization (being detached from one-self).

12. Fear of losing control or “going crazy.”

13. Fear of dying.

Thus, at least 4, 5, 6, 7, 8, 9, 10, 11 or 12 of these symptoms can occur during a panic attack.

Panic Disorder: Recurrent unexpected panic attacks (see Criterion A, below). A panic attack is an abrupt surge of intense fear or intense discomfort that reaches a peak within minutes, and during which time four or more of a list of 13 physical and cognitive symptoms occur. The term recurrent means more than one unexpected panic attack. The term unexpected refers to a panic attack for which there is no obvious cue or trigger at the time of occurrence so that the attack appears to occur without warning, such as when the individual is relaxing or emerging from sleep (nocturnal panic attack). In contrast, expected panic attacks are attacks for which there is an obvious cue or trigger, such as a situation in which panic attacks typically occur. The determination of whether panic attacks are expected or unexpected is made by the clinician. Approximately one-half of individuals with panic disorder have expected panic attacks as well as unexpected panic attacks, so that the presence of expected panic attacks does not rule out the diagnosis of panic disorder. The frequency and severity of panic attacks vary widely. In terms of frequency, there may be moderately frequent attacks (such as one per week or per ten days) for months at a time, or short bursts of more frequent attacks (such as daily or twice daily) separated by weeks or months without any attacks or with less frequent attacks (such as two per month) over many years. In terms of severity, individuals with panic disorder may have both full-symptom (four or more symptoms) and limited-symptom (fewer than four symptoms) attacks, and the number and type of panic attack symptoms frequently differ from one panic attack to the next. However, more than one unexpected full-symptom panic attack is required for the diagnosis of panic disorder.

The worries about panic attacks or their consequences usually pertain to physical concerns, such as worry that panic attacks reflect the presence of life-threatening illnesses (e.g., cardiac disease, seizure disorder); social concerns, such as embarrassment or fear of being judged negatively by others because of visible panic symptoms; and concerns about mental functioning, such as “going crazy” or losing control (Criterion B, see below). A panic disorder is characterized by the following features:

A. Recurrent unexpected panic attacks.

B. At least one of the attacks has been followed by 1 month (or more) of one or both of the following:
1. Persistent concern or worry about additional panic attacks or their consequences; and
2. A significant maladaptive change in behavior related to the attacks (such as, but not limited to, behaviors designed to avoid having the panic attacks).

C. The disturbance is not attributable to the physiological effects of a substance (for example, a drug of abuse, a medication) or another medical condition (such as, but not limited to, hyperthyroidism or cardiopulmonary disorders).

D. The disturbance is not better explained by another mental disorder, such as a social anxiety disorder; a specific phobia; obsessive-compulsive disorder; posttraumatic stress disorder; or separation anxiety disorder.

Post-Traumatic Stress Disorder (PTSD): A disorder that can occur after experiencing a traumatic event that leaves a subject feeling scared, confused, and/or angry to the extent that daily activities are difficult to perform. A traumatic event can include combat or military exposure, child sexual or physical abuse, terrorist attacks, sexual or physical assault, serious accidents, and natural disasters (such as a fire, tornado, hurricane, flood, or earthquake). PTSD is defined by the Diagnostic and Statistical Manual (DSM), Fourth-Edition, Text Revision, published by the American Psychiatric Associating (DSM-IV-TR), and the DSM-V.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Psychiatric disorder: Any disorder that results in altered or abnormal behavior, function, or subjective distress in one or more of the following intrapersonal or interpersonal realms: mood, anxiety, memory, cognition, consciousness, perception, sexual experience, sleep, substance use/addiction, personality, attention/concentration, psychosis, identity, eating, or bodily function or integrity. A psychiatric disorder typically causes the patient or others around the patient noticing social, interpersonal, and/or occupational distress or dysfunction. The cause (etiology) of a psychiatric disorder may be idiopathic (unknown), or it may be due to a recognized psychosocial stressor, a medical disorder, or a neurological disorder.

Psychotic: A psychiatric condition in its broadest sense, as defined in the DSM-IV (Kaplan, ed. (1995) supra). Different disorders which have a psychotic component comprise different aspects of this definition of “psychotic.” For example, in schizophreniform disorder, schizoaffective disorder and brief psychotic disorder, the term “psychotic” refers to delusions, any prominent hallucinations, disorganized speech, or disorganized or catatonic behavior. In psychotic disorder due to a general medical condition and in substance-induced psychotic disorder, “psychotic” refers to delusions or only those hallucinations that are not accompanied by insight. Finally, in delusional disorder and shared psychotic disorder, “psychotic” is equivalent to “delusional” (see DSM-IV, supra, page 273).

Objective tests can be used to determine whether an individual is psychotic and to measure and assess the success of a particular treatment schedule or regimen. For example, measuring changes in cognitive ability aids in the diagnosis and treatment assessment of the psychotic patient. Any test known in the art can be used, such as the so-called “Wallach Test,” which assesses recognition memory (see below, Wallach, J. Gerontol. 35:371-375, 1980). Another example of an objective text that can be used to determine whether an individual is psychotic and to measure efficacy of an anti-psychotic treatment is the Stroop Color and Word Test (“Stroop Test”) (see Golden, C. J., Cat. No. 30150M, in A Manual for Clinical and Experimental Uses, Stoelting, Wood Dale, Ill.). The Stroop Test is an objective neuropsychiatric test that can differentiate between individuals with psychosis and those without.

Psychosis: A psychiatric symptom, condition or syndrome in its broadest sense, as defined in the DSM-IV (Kaplan, ed. (1995) supra), comprising a “psychotic” component, as broadly defined above. Psychosis is typically defined as a mental disorder or condition causing gross distortion or disorganization of a person's mental capacity, affective response, and capacity to recognize reality, communicate, and relate to others to the degree of interfering with his capacity to cope with the ordinary demands of everyday life.

Psychotic major depression: A condition also referred to as psychotic depression (Schatzberg, Am. J. Psychiatry 149:733-745, 1992), “psychotic (delusional) depression,” “delusional depression,” and “major depression with psychotic features” (see the DSM-III-R). This condition is a distinct psychiatric disorder that includes both depressive and psychotic features. Individuals manifesting both depression and psychosis, i.e. psychotic depression, are often referred to as “psychotic depressives.”

Seizure Disorder: A “paroxysmal event due to abnormal, excessive, hypersynchronous discharges from an aggregate of central nervous system (CNS) neurons” that may or may not result in observable changes in behavior (Chapter 363 of Harrison's Principles of Internal Medicine (Fauci A S, Kasper D L, et al. (editors), 17^{th}Edition, 2008). A seizure is a single event while epilepsy or seizure disorder is a medical diagnosis to describe a condition characterized by repeated seizures. Various types of seizures include simple partial, complex partial, partial with secondary generalization, absence, atypical absence, generalized tonic-clonic, atonic, myoclonic, or unclassified. Brain injury as defined above is a recognized cause of seizures. Seizures can be associated with various additional clinical problems: cognitive changes, mood or anxiety changes, interictal behavior changes, sudden death, psychosocial impairments, occupational problems, or psychosis.

Scowl: A facial expression showing displeasure. Scowling primarily utilizes the corrugator supercilii muscle and the procerus muscle.

Season: A particular time of year. Generally, the year is divided into four seasons, fall, winter, spring, and summer. The exact dates for each season can be determined for any calendar year. Seasons are determined by the position of the sun relative to the earth.

Seasonal Affective Disorder: The Diagnostic and Statistical Manual (DSM), Fourth-Edition, Text Revision (DSM-IV-TR) classifies SAD as a subtype or “course specifier” for recurrent major depressive episodes within major depressive disorder or bipolar disorder (BD). However, only a minority of patients with depression or BD are diagnosed as having BD. SAD can be diagnosed by the following criteria:

A. A person has experienced a regular pattern of depressive episodes that begin at specific time of the year (e.g., fall or winter), and which are not related to specific yearly stressors such as school/college or seasonal unemployment.

B. The depression also ends (full remission) or changes to mania at a specific time of the year (e.g., spring).

C. The pattern has occurred for the most recent two years with no other symptoms outside of the pattern.

D. A person has had more seasonal depressions than non-seasonal depressions in his/her lifetime.

Selective Serotonin Reuptake Inhibitor (SSRI): A type of antidepressant medication that is prescribed for the treatment of various psychiatric conditions, including, but not limited to, a depressive disorder or an anxiety disorder. Commonly prescribed SSRIs include fluoxetine, paroxetine, sertraline, citalopram, escitalopram, and fluvoxamine. Other non-limiting examples of SSRI include prodrug or pharmacologically active metabolite of these SSRI medications.

Sertraline: A selective serotonin reuptake inhibitor that is prescribed to treat one or more of the following indications: major depression or a depressive disorder, OCD, PTSD, panic disorder, social phobia, PMDD, or an anxiety disorder.

Sign: An observation, result, finding, or outcome on a medical test or examination that may indicate the presence of an associated medical, neurologic, or psychiatric condition. Non-limiting examples include observed behavior reported by a non-medical observer (e.g., family member, friend, law enforcement officer, clergy, fellow member of a military unit); observed behaviors during clinical evaluation such as depression noted on mental status examination; psychological or neuropsychological test results; laboratory value from blood, urine, cerebrospinal fluid; radiologic examinations such as x-rays, CT or MR scans; physical examination results such as impaired coordination or disconjugate eye movements on neurological examination, or elevated blood pressure on physical examination; or oculomotor function on vestibulo-oculomotor examination.

Sleep disorder: A disorder of sleep that includes, but is not limited to, insomnia, disorders of daytime somnolence, parasomnias, chronobiological disorders, and sleep consequences of neurological disorders. Non-limiting examples of sleep disorders include rapid eye movement behavior disorder, restless legs syndrome, periodic leg movements of sleep, obstructive sleep apnea, central sleep apnea, nightmares, sleep terrors, sleepwalking, confusional arousals, sleep paralysis, sleep eating disorder, or narcolepsy (See, for example, C G Goetz (editor), Textbook of Clinical Neurology, 3rd Edition, 2007, Chapter 54)

Sleep disturbance: An observed or reported alteration in the initiation, maintenance, or quality of sleep that may be a symptom or sign of a medical, neurological, or psychiatric disorder. A sleep disturbance also may be a symptom or sign of a sleep disorder.

Social Anxiety Disorder: According to the Diagnostic and Statistical Manual of Mental Disorders (5^{th} Ed., American Psychiatric Association, Arlington, Va., 2013), a social anxiety disorder is characterized by:
A. Marked fear or anxiety about one or more social situations in which the individual is exposed to possible scrutiny by others. Examples include social interactions (e.g., having a conversation, meeting unfamiliar people), being observed (such as eating or drinking), and performing in front of others (such as giving a speech). In children, the anxiety must occur in peer settings and not just during interactions with adults.
B. The individual fears that he or she will act in a way or show anxiety symptoms that will be negatively evaluated (i.e., will be humiliating or embarrassing; will lead to rejection or offend others).
C. The social situations almost always provoke fear or anxiety. In children, the fear or anxiety may be expressed by crying, tantrums, freezing, clinging, shrinking, or failing to speak in social situations.
D. The social situations are avoided or endured with intense fear or anxiety.
E. The fear or anxiety is out of proportion to the actual threat posed by the social situation and to the sociocultural context.
F. The fear, anxiety or avoidance is persistent, typically lasting for six months or more.
G. The fear, anxiety or avoidance causes clinically significant distress or impairment in social, occupational, or other important areas of functioning.
H. The fear, anxiety or avoidance is not attributable to the physiological effects of a substance (e.g., a drug of abuse, a medication) or another medical condition.
I. The fear, anxiety or avoidance is not better explained by the symptoms of another mental disorder, such as panic disorder, body dysmorphic disorder, or autism spectrum disorder.
J. If another medical condition (e.g., Parkinson's disease, obesity, disfigurement from burns or injury) is present, the fear, anxiety, or avoidance is clearly unrelated or is excessive.

Subject: Any mammal. In one embodiment, a subject is a human subject.

Symptom: A problem, complaint, or issue reported by a subject that is primarily a subjective complaint. Pain, fatigue, or changes in mood are commonly reported symptoms. Symptoms are distinguished from signs in that signs typically can be confirmed with objective evidence such as observation, tests or examinations, whereas symptoms rely upon the subject's self-report.

Therapeutically effective amount: A quantity of treatment, such as drug, for example a neurotoxin such as Botulinum toxin A, sufficient to achieve a desired effect in a subject being treated. For instance, this can be the amount of Botulinum toxin A necessary to impair contraction of, or paralyze, a facial muscle. This can also be the amount of a therapy, such as light therapy or psychotherapy, sufficient to relieve a symptom of a disorder, such as depression. This can also be the amount of an antidepressant sufficient to alter the mood of a subject.

Unit equivalents: An amount of Botulinum Toxin (BTX) that is equivalent to standard Units of Botulinum Toxin A (BTX-A). A standard Unit of BTX-A is defined as the mean LD₅₀ for female Swiss Webster mice weighing 18-20 grams (Schantz and Kaultner, J. Assoc. Anal. Chem. 61: 96-99, 1978). The estimated human LD₅₀ for a 70-kg person is 40 Units/kg or about 2500-3000 Units.

BOTOX™ (Botulinum toxin A, Allergan, Inc., Irvine, Calif., U.S.A.) is sold in 100 Unit vials. DYSPORT™ (Speywood Pharmaceuticals, Ltd., Maidenhead, U.K.) is sold in 300 or 500 Unit vials. For cosmetic uses, the vial contents are typically diluted with 1 or 2 ml of sterile saline solution, which for BOTOX™ provides a 100 or 50 Unit/ml dilution. DYSPORT™ BTX-A is roughly three fold less toxic than BOTOX™ and approximately three-fold greater amounts of the DYSPORT™ product will usually be injected to achieve the same result as would be obtained using a specific number of Units of BOTOX™.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Botulinum Toxin

Botulinum toxin (BTX), produced by the bacterium Clostridium botulinum reversibly paralyzes striated muscle when administered in sub-lethal doses. BTX has been used in the treatment in a number of neuromuscular disorders and conditions involving muscular spasm including, but not limited to, dystonia, hemifacial spasm, tremor, spasticity (e.g. resulting from multiple sclerosis), anal fissures and various ophthalmologic conditions (for example, see Carruthers et al., J. Amer. Acad. Derm. 34:788-797, 1996). A Botulinum toxin type A complex has been approved by the U.S. Food and Drug Administration for the treatment of blepharospasm, strabismus and hemifacial spasm.

BTX is a generic term covering a family of toxins produced by C. botulinum comprising up to eight serologically distinct forms (A, B, C₁, C₂, D, E, F and G). These toxins are among the most powerful neuroparalytic agents known (c.f. Melling et al., Eye 2:16-23, 1988). Serotypes A, B and F are the most potent. Without being bound by theory, the mode of action is believed to be an inhibition of the release of acetylcholine by the presynaptic nerve.

Botulinum toxin type A can be obtained by establishing and growing cultures of Clostridium botulinum in a fermenter and then harvesting and purifying the fermented mixture in accordance with known procedures. Alternatively, the Botulinum toxin serotypes are initially synthesized as inactive single chain proteins which must be cleaved or nicked by proteases to become neuroactive. High quality crystalline Botulinum toxin type A can be produced from the Hall A strain of Clostridium botulinum. The Schantz process can be used to obtain crystalline Botulinum toxin type A (see Schantz et al., Microbiol Rev. 56:80-99, 1992). Generally, the Botulinum toxin type A complex can be isolated and purified from an anaerobic fermentation by cultivating Clostridium botulinum type A in a suitable medium. This process can be used, upon separation out of the non-toxin proteins, to obtain pure Botulinum toxins, such as for example: purified Botulinum toxin type A with an approximately 150 kD molecular weight, purified Botulinum toxin type B with an approximately 156 kD molecular weight and purified Botulinum toxin type F with an approximately 155 kD molecular weight.

Botulinum toxins and/or Botulinum toxin complexes can be obtained from List Biological Laboratories, Inc., Campbell, Calif.; the Centre for Applied Microbiology and Research, Porton Down, U.K.; Wako, Osaka, Japan; Metabiologics, Madison, Wis.) as well as from Sigma Chemicals, St Louis, Mo. There are several formulations that have been approved by the U.S. Food and Drug administration, and any of these can be used in any of the methods disclosed herein. These include BOTOX®, DYSPORT®, XEOMIN®, and MYOBLOC®.

The initial cosmetic use of BTX was for treatment of forehead frown lines as reported in Carruthers and Carruthers, J. Dermatol. Surg. Oncol. 18:17-21, 1992. The clinical effects of peripheral intramuscular Botulinum toxin type A are usually seen within one week of injection. The typical duration of symptomatic relief from a single intramuscular injection of Botulinum toxin type A averages about three months. BTX-A serotype is available commercially for pharmaceutical use under the trademarks BOTOX™ (Allergan, Inc., Irvine, Calif., U.S.A.) and DYSPORT™ (Speywood Pharmaceuticals, Ltd., Maidenhead, U. K.). BOTOX™ consists of a purified Botulinum toxin type A complex, albumin and sodium chloride packaged in sterile, vacuum-dried form. Specifically, each vial of BOTOX™ contains about 100 Units (U) of Clostridium botulinum toxin type A purified neurotoxin complex, 0.5 milligrams of human serum albumin and 0.9 milligrams of sodium chloride in a sterile, vacuum-dried form without a preservative.

The Botulinum toxin type A is made from a culture of the Hall strain of Clostridium botulinum grown in a medium containing N-Z amine and yeast extract. The Botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C.

BOTOX™ is sold in 100 Unit vials. DYSPORT™ is sold in 300 or 500 Unit vials. BTX-A is roughly three-fold less toxic than BOTOX™ and approximately three-fold greater amounts of the DYSPORT™ product will usually be injected to achieve the same result as would be obtained using a specific number of Units of BOTOX™.

For cosmetic uses, the vial contents are typically diluted with 1 or 2 ml of sterile saline solution, which for BOTOX™ provides a 100 or 50 Unit/ml dilution. (DYSPORT™ can also be utilized.) For example, BOTOX™ can be reconstituted with sterile, non-preserved saline prior to intramuscular injection. To reconstitute vacuum-dried BOTOX™, sterile normal saline without a preservative (0.9% Sodium Chloride Injection) is used by drawing up the proper amount of diluent in the appropriate size syringe. Since BOTOX™ may be denatured by bubbling or similar violent agitation, the diluent is gently injected into the vial. For sterility reasons BOTOX™ is preferably administered within four hours after the vial is removed from the freezer and reconstituted. During these four hours, reconstituted BOTOX™ can be stored in a refrigerator at about 2° C. to about 8° C. Reconstituted, refrigerated BOTOX™ has been reported to retain its potency for at least about two weeks (see Neurology, 48:249-53:1997).

### Methods for Treatment of Seasonal Affective Disorder (SAD)

Methods are provided herein for treating or preventing SAD in a subject. The SAD is caused by the onset of a specific season. In specific, non-limiting examples, the season is winter. The method includes administering a therapeutically effective amount of a neurotoxin to a facial muscle involved in frowning, scowling, or a sad appearance. The agent causes partial or complete paralysis of the facial muscle, thereby affecting the ability of the subject to frown and thereby treat SAD.

In some embodiments, the SAD is prevented, which means it does not occur in a clinically recognizable manner in the season that immediately follows the administration of the neurotoxin. Thus, preventing indicates that the SAD does not occur during a period during which the neurotoxin has functionally de-enervated the target muscles into which it was injected. Typically, the effects of the neurotoxin can last about 3 months and will therefore be effective for much of a season during which SAD normally occurs, such as winter. Injections can be repeated as necessary to prolong the effect for a period of time that corresponds to that season or seasons. Injections can be discontinued in seasons where SAD does not occur. In some examples, SAD does not occur within a year of administration of the neurotoxin. In some non-limiting examples, the subject does not have SAD in the winter following the administration of the neurotoxin. The subject can be treated, meaning objective improvement in one or more of the symptoms of the SAD is achieved during the season. In some non-limiting examples, one or more symptoms of SAD is improved, or one or more of the symptoms of SAD is absent in the season, such as winter.

The injection sides for treatment include sites in the glabella, such as, but not limited to, the procerus muscle and/or the corrugator supercilii muscle. In some embodiments a therapeutically effective amount of BTX, such as BTX A, is administered to the procerus muscle and the corrugator supercilii muscle. In some embodiments, BTX is not injected into the orbicularis oculi, frontalis, and/or the depressor anguli oris muscles (trianglaris muscle). In other embodiments, BTX is injected into the depressor anguli oris muscles (trianglaris muscle).

The method involves identifying or selecting subjects who have SAD, or who have SAD in previous years, and administering the treatment to these subjects. The subject can be female or male. The Diagnostic and Statistical Manual (DSM), Fourth-Edition, Text Revision (DSM-IV-TR) classifies SAD as a subtype or “course specifier” for recurrent major depressive episodes within major depressive disorder or bipolar disorder (BD). However, only a minority of patients are diagnosed as having BD. SAD can be diagnosed by the following criteria:
A. A person has experienced a regular pattern of depressive episodes that begin at specific time of the year (e.g., fall or winter), and which are not related to specific yearly stressors such as school/college or seasonal unemployment.
B. The depression also ends (full remission) or changes to mania at a specific time of the year (e.g., spring).
C. The pattern has occurred for the most recent two years with no other symptoms outside of the pattern.
D. A person has had more seasonal depressions than non-seasonal depressions in his/her lifetime.
   A subject with SAD, for example, can notice that in the winter months they have a lack of energy or feel sluggish, they sleep more than usual, they overeat and gain weight, and they may have a craving for carbohydrates. The subject can be a woman and/or live at a high altitude, which are risk factors for SAD. Thus, the subject can have a mood disorder that is seasonal (MDD SP).

Bipolar I disorder (BD I) or bipolar II disorder (BD II) with seasonal pattern (BD SP) is the DSM-IV-TR diagnosis for persons with depressive episodes in the fall or winter and mania (BD I) or hypomania (BD II) in spring or summer. It has been postulated that circadian phase delays, in which internal rhythms lag behind the sleep cycle, can cause all of the types of SAD. It has also been postulated that a difference in nightly melatonin release, due to the difference in the length of days/nights, can cause SAD.

A subject can be selected that has SAD as defined by the DSM-IV-TR criteria. A subject can also be selected that has SAD as defined by the DSM-V criteria. A subject can be selected who has bi-polar disorder with seasonal depression (BD-SP). The subject can be an adult or a child. The subject can be a woman or a man. The subject can have experienced SAD for at least two years, such as 3, 4, 5, 6, 7, 8, 9, 10 or more years. In some embodiments, the subject has SAD, but does not have any other psychiatric disorder.

A subject is selected that has SAD. In some embodiments, the subject does not have an underlying physical condition that is being treated by BTX, such as a musculoskeletal condition, such as torticollis, blepharospasm, or other disorder of muscular contractions. In other embodiments, the subject does not have a muscular disorder or condition. In some examples, the subject does not have spasms, cramping, and tightness of muscles cause by musculoskeletal conditions/disease. In additional embodiments, the subject has (or does not have) wrinkles, and is not being treated using Botulinum toxin for any cosmetic purposes, including the treatment of wrinkles. In some embodiments, the subject has a comorbid condition.

In further embodiments, the subject does not have other psychological conditions. In some examples, the subject does not have an acute stress disorder or an anxiety disorder. In further embodiments, the subject does not have other forms of depression, such as major depression or a mood disorder. In additional embodiments, the subject does not have post-traumatic stress disorder, obsessive compulsive disorder, panic disorder, a sleep disorder, a mood disorder, or a social anxiety disorder.

Thus, in some embodiments, SAD is the sole psychological disorder; the subject does not have, for example schizophrenia, depression, other anxiety disorders, schizophrenia, mania and/or obsessive compulsive disorder. In some embodiments, the subject does not have a psychotic disorder. In additional embodiments, the subject does not have a sleep disorder, sleep terrors and/or a sleep disturbance cause by any factor other than SAD.

The neurotoxin, such as BTX, for example BTX A can be administered prior to the season of onset of the SAD. The BTX can be administered, for example, about two months to about two weeks prior to the season. The BTX can be administered about 1, 2, 3, 4, 5, 6, 7, 8, 9, or 10 weeks prior to the season. The BTX can be administered 2 to 6 weeks prior to the season. The BTX can be administered only a single time prior to the season. In other embodiments, the BTX is administered 1, 2, or 3 times prior to the season. The treatment can be repeated in the season preceding the onset of SAD when the partial or complete muscle paralysis induced by the agent begins to abate In addition, the BTX can be administered during the season, such as 1, 2, or 3 times during the season. The treatment can be repeated in the season preceding the onset of SAD when the partial or complete muscle paralysis induced by the agent begins to abate.

In further embodiments, BTX is not administered during the seasons were the subject does not experience SAD. Thus, administration is limited to a number of administrations immediately prior to, or during, the season of onset. For example, if the subject experiences SAD in season 2, the subject is administered SAD only 1, 2, or 3 times in season 1 (preceding season 2), and/or 1, 2 or 3 times during season 2 (the season wherein the subject experiences SAD). The subject is not administered BTX in seasons 3 and 4.

In another example, if the subject experiences SAD in the winter, the subject is administered SAD only 1, 2, or 3 times in the fall, and/or 1, 2 or 3 times during the winter. The subject is not administered BTX in the spring and the summer. In one specific non-limiting example, the subject is only administered one or two times prior to the onset of the winter (such as in the fall), and once in the winter, and the subject is not administered BTX in the spring or the summer. In another specific non-limiting example, the subject is only administered one or two times prior to the onset of the winter (such as in the fall), and the subject is not administered BTX in the winter, spring or the summer. One of skill in the art can readily apply these treatment protocols for other seasons. For example, if the season of onset is the summer, the BTX can be used in the spring and summer, but not in the fall or winter.

An exemplary injection technique involves the use of a short, narrow needle (e.g. ½ inch or 8 mm; 30-gauge) with an insulin- or tuberculin-type syringe. Subjects are typically treated in the seated position. The skin area is cleaned with an alcohol swab. A single syringe may be used for multiple injections to treat different locations in a single muscle or more than one muscle. Typically, the plunger of the syringe is depressed as the needle is withdrawn so that toxin is evenly distributed at the injection site. Pressure or gentle massage may be applied at the injection site to assist in dissipating the toxin. The toxin will typically migrate approximately 1 cm from the site of injection. Without being bound by theory, the toxin (such as BTX A) does not cross the blood brain barrier.

Electromyographic (EMG) guided needles may be used for injection to determine needle location of a high degree of accuracy, although this technique is generally not necessary.

For applications of BTX, such as BTX A, total dose per treatment can be varied and depends upon the condition being treated and the site of application of BTX. For example, a total dose of about 10 to about 60 Units, such as about 20 to about 50 Unity equivalents, about 30 to about 60 Units, about 29 to about 40 units, about 30 to about 40 units, or about 20 to about 40 Unit equivalents. The BTX, such as BTX A will typically be applied to corrugator supercilii and the procerus muscle (see, Carruthers and Carruthers, Dermatol. Surg. 24:1168-1170, 1998 for dosing information).

In some embodiments, BTX, such as BTX A, is not administered to the orbicularis oculi, frontalis, and/or the depressor anguli oris muscles (trianglaris muscle). In one specific non-limiting example, about 20 to about 50 Unit equivalents of BTX A, such as 30 to 40 Unit Equivalents of BTX, is administered only to the corrugator supercilii and/or the procerus muscle or both. In specific embodiments, about 29, 30, 31, 32, 33, 34, 45, 36, 37, 38. 39 or 40 Unit Equivalents of BTX is administered. The administration can occur 1, 2, 3, 4, or 5 times. In some embodiments, only a single administration is utilized.

In further embodiments, a total dose of about 20 to about 60 Unit equivalent of BTX are administered to the corrugator supercilli, procerus muscle and the depressor angularis oris muscle. In some non-limiting examples, a total of about 28 to about 58 Unit equivalants of BTX are administered to the corrugator supercilli, procerus muscle, and depressor angularis oris muscles, wherein about 4 Unit equivalents are administered to the left depressor angularis oris muscle and to the right depressor angularis oris muscle.

Onset of muscle paralysis following injection usually occurs within days of treatment, although it can take up to ten days for full paralysis to occur. In some embodiments, it can take one or two days for relief of symptoms, but it may take longer, such as a week or two weeks for improvement. The duration of paralysis will vary from patient to patient. Typically, duration will be from two to Six months, for example about three to about six months, or for example about three months, before subsequent treatment is required to alleviate the condition, although BTX occasionally has efficacy for up to 12 months (Naumann et al., European J. Neurology 6(Supp 4):S111-S115, 1999). In some embodiments, the BTX is administered yearly, prior to the onset of the season associated with SAD, using any of the protocols listed above.

In additional embodiments, the methods disclosed herein are utilized concurrently with light therapy, cognitive behavioral therapy and/or psychotherapy. In additional embodiments, BTX, such as BTX A, is administered in conjunction with other agents, such as for the treatment of depression. The treatment can be light therapy and/or a pharmaceutical agent for the treatment of depression. In some embodiments, the subject is not administered an additional pharmaceutical agent, such as chemical compounds, for the treatment of depression. In addition embodiments, the BTX, such as BTX A, is administered in conjunction with light therapy, either before, after or during light therapy.

Light therapy, specifically exposure to bright light, has been found to be an effective means of treating seasonal affective disorder. The individual sits in front of a bright light unit, which is a specialized, portable box which houses balanced spectrum fluorescent tubes. An individual's needs for light therapy specifies the duration of exposure and the optimal time of day. The most common device used for bright light therapy is a fluorescent light box which produces a light intensity of 2,500 to 10,000 lux at a comfortable distance (such as about ½ foot to 2.5 feet from a subject, for example, about 1 to about 2 feet from the subject, such as 1, 1.5, or 2 feet from the subject). Light box intensity measurements, as advertised by the manufacturers, have been calibrated at the specific distance.

Without being bound by theory, decreased movement of muscles of a neural circuit that is involved in SAD lessen the symptoms of SAD. Denervation of the frown muscles results in a decreased ability of the treated subject to frown, and thus inhibits fear, anger and sadness, contributing to a subjective sense of a different season. Improvement can be objectively assessed by the DSM-IV or DMSV-V criteria, or by standardized tests known in the art. As noted above, SAD can be assessed, for example, by the Beck Depression Inventory II (1996, Harcourt). A very straightforward test is to ask the subject to report whether the intensity of the symptoms has changed.

Full-spectrum light is not necessary since intensity is most important, but a balanced-spectrum light minus UV-B emissions is recommended in light therapy that can accompany de-enervation using a neurotoxin such as BTX. Bright light therapy can be administered at any time of day, but is often most effective within the first two hours of waking in the morning. In some embodiments, light therapy is administered daily, such as for about 30 to about 90 minutes, such as for about 45 minutes to about 60 minutes, about 45 to about 90 minutes, or for about 60 to about 90 minutes. Exemplary times include, for example, about 30, about 35, about 40, about 45, about 50, about 55, about 60, about 65, about 70, about 75, about 80, about 85 or about 90 minutes.

In other embodiments, the subject is administered additional anti-depressant medication, including, but not limited to SSRIs, such as citalopram, escitalopram, fluoxetine, fluvoxamine, paroxetine, and sertraline, and/or MAOIs. In further embodiments, Botulinum toxin is administered in conjunction with an alpha-adrenergic antagonist, such as clonidine, an anti-convulsant or mood stabilizers (for example, carbamazepine (TERGRETOL®), Topiramate (TOPAMAX®), Zolpidem (AMBIEN®), Lamotrigine (LAMICTAL®), Valproic acid (DEPAKENE®), lithium carbonate, buspirone (BUSPAR®), and alpha adrenergic blockers such as Prazosin (MINIPRESS®)). In additional embodiments, Botulinum toxin is administered with an antipsycholic agent, such as risperidone, or an atypical antidepressant, a beta blocker (such as propanolol), a benzodiazepine, a glucocorticoid (such as cortiosterone), a heterocyclic antidepressant, such as amitriptyline, or imipramine, or a MAOI, such as phenelzine.

The BTX, such as BTX A, can be administered simultaneously or sequentially with the additional modality of treatment. In one embodiment, a therapeutically effective amount of BTX, such as BTX A, is administered in combination with at least one additional modality of treatment for SAD. Useful modalities are listed above. The modality of treatment can be physical, such as electroconvulsive therapy, light therapy, or electromagnetic radiation. The modality can also be psychotherapy, exercise or meditation. A single modality of treatment can be combined with the BTX treatment, or a combination of additional modalities can be used with the BTX treatment. Thus, in one example, a subject taking a therapeutically effective amount of an antidepressant medication (such as an SSRI), or undergoing a therapeutic protocol, can be treated with BTX, such as BTX A, during the course of the additional treatment, such as light therapy.

The subject can be administered an additional therapeutic agent, such as a therapeutically effective amount of one or more of: a Selective Serotonin Reuptake Inhibitor (SSRI), an alpha adrenergic antagonist, an anti-convulsant, a mood stabilizer, an anti-psychotic agent, a beta blocker, a benzodiazepine, a glucocorticoid, a monamine-oxidase inhibitor (MAOIs), a heterocyclic anti-depressant, a tricyclic anti-depressant and/or an atypical anti-depressant.

BTX, such as BTX A, can be administered after the treatment with the additional agent has been terminated, or prior to the initiation of therapy, such as the administration of the antidepressant medication, psychotherapy, or a physical treatment protocol, such as light therapy. Thus, the treatment can be simultaneous. The treatment can be sequential, so that the other modality of treatment is administered before BTX, after BTX, or in other seasons that the BTX. In other embodiments, BTX is administered in the absence of treatment with these agents.

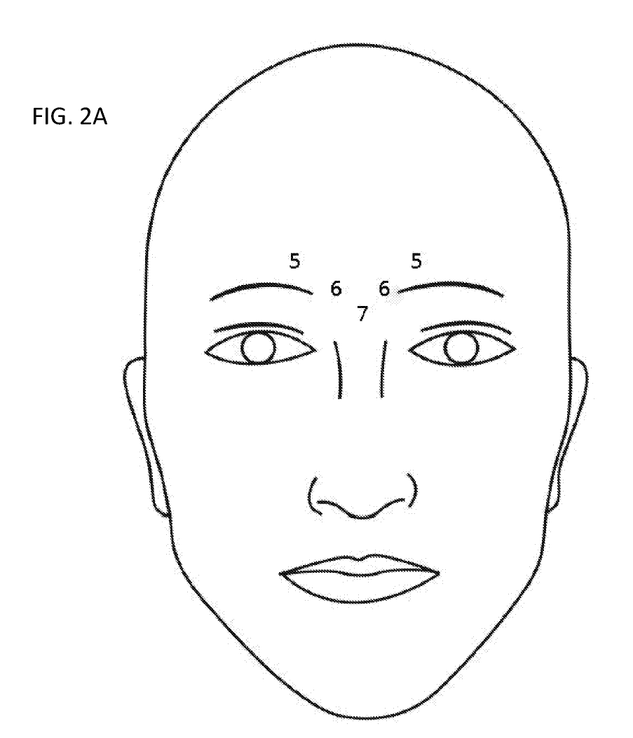

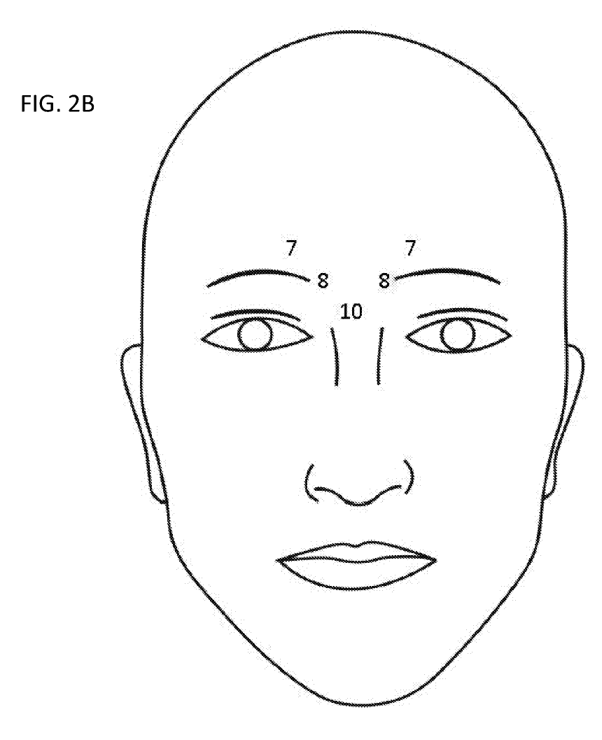

FIGS. 2A-2B show an exemplary Botulinum toxin dose used to treat the frown in a typical woman (FIG. 2A) and man (FIG. 2B). The landmarks for the injection are as follows (see also Carruthers et al., Derm. Surg. 24:1189-1194, 1998). The injection of the procerus is below a line joining the medial end of both eyebrows. The landmark for the next injections is a line vertically above the inner canthus and the superior margin of the orbit. Botulinum toxin is injected just superior to the first injection point. Next an injection is made 1 centimeter (cm) medial and one-half cm superior to the second two injections. The numbers refer to the number of BTX Units injected in the area shown.

The disclosure is illustrated by the following non-limiting Examples.

### EXAMPLES

The following examples document treatment of SAD by administering a neurotoxin, such as BTX A, to the glabella, for example into a corrugator supercilli and/or a procerus muscle of the subject.

### Example 1

### Case Study 1

K. M. is a 47 year old man with a strong history of severe fall and winter depressions for the previous 8 years. He was free of depression during the summer and met criteria for seasonal affective disorder. Initially, the depressions, which began in November and lasted several months, responded well to light therapy alone. After the first winter, however, it was necessary to add antidepressants to the light therapy. However, in the last two to three winters this combination did not fully reverse his winter depressive symptoms.

Anticipating the next winter, he was administered botulinum toxin A treatment by glabella injection, which was initiated in October, with a dose of 40 units. He had no depressive symptoms whatsoever during the ensuing winter and spontaneously reported that the addition of botulinum toxin A treatment to the regimen, that had previously failed to forestall or treat his SAD symptoms, had enabled him to enjoy a depression free winter for the first time in years.

### Example 2

### Case Study 2

S. F. is a 56 year old artist and writer who suffered from seasonal affective disorder since adolescence. Her depressions typically lasted 4 months (from December to March) and were treated successfully with a combination of antidepressant medications and light therapy. In the last few winters, however, antidepressants had to be discontinued because of unacceptable side effects, so she had to rely on light therapy alone which was insufficient to treat her depression.

Before the last winter, the patient began to receive botulinum toxin A treatments to the glabella, 29 units, every 3 months in an attempt to forestall her depression. The treatment succeeded, and the patient did not become depressed in the ensuing winter, which was the first time that had occurred since she stopped using antidepressant medications.

### Example 3

### Case Study 3

E. R. is a 65 year old physician who has suffered from seasonal affective disorder for 40 years. He treated this recurrent illness with light therapy accompanied by significantly reducing his stress in the winter. On multiple occasions, whenever he has a significant stress, especially when he was under deadline pressures, he would experience the classic symptoms of seasonal affective disorder notwithstanding the use of light therapy. He tried antidepressant medication on a few occasions but found their side effects to be unacceptable and therefore these medications were of no use to him. Because of the close association between deadline pressures and the emergence of depressive symptoms, E. R. diligently avoided such stresses in the wintertime.

On entering the last winter, however, E. R. was confronted by delays in a publication process that he could not have foreseen. Realizing that these pressures would inevitably precipitate his seasonal affective disorder symptoms, E. R. received botulinum toxin A treatments to the glabella, 40 units, in December, in advance of the time his symptoms would normally occur. E. R. had no seasonal affective disorder symptoms in the ensuing winter, during which he was exceptionally productive. He said, “After getting the botulinum toxin A injections it was as though the winter was completely neutralized as far as the mood was concerned.”

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for treating or preventing seasonal affective disorder caused by the onset of a specific season in a subject, comprising administering a therapeutically effective amount of a neurotoxin to a facial muscle to cause paralysis of the facial muscle, thereby affecting the ability of the subject to frown prior to the onset of the season during which the seasonal affective disorder occurs, thereby treating or preventing seasonal affective disorder in the subject.

2. The method of claim 1, wherein the neurotoxin is administered to a glabella of the subject.

3. The method of claim 1, wherein the neurotoxin is administered to a corrugator supercilli and a procerus muscle of the subject.

4. The method of claim 1, wherein the subject does not have major depression, depression extending throughout the entire calendar year, an obsessive compulsive disorder, a panic disorder, a social anxiety disorder, a post-traumatic stress disorder, and/or an underlying skeletal muscle disorder.

5. The method of claim 4, wherein the skeletal muscle disorder is torticollis.

6. The method of claim 1, wherein the neurotoxin is a Botulinum toxin.

7. The method of claim 6, wherein the Botulinum toxin is Botulinum toxin A.

8. The method of claim 7, wherein about 30-50 Unit equivalents of Botulinum toxin type A is administered to the corrugator supercilli and the procerus muscle of the subject.

9. The method of claim 7, wherein about 29-40 Unit equivalents of Botulinum toxin type A is administered to the corrugator supercilli and the procerus muscle of the subject.

10. The method of claim 8, further comprising administering an additional dose of about 30-50 Unit equivalents of Botulinum toxin type A to the glabella after about two to three months.

11. The method of claim 9, further comprising administering an additional dose of about 30-40 Unit equivalents of Botulinum toxin type A to the glabella after about two to three months.

12. The method of claim 8, wherein the Botulinum toxin type A is administered about two months to about two weeks prior to the onset of the season during which the seasonal affective disorder occurs.

13. The method of claim 8, wherein the Botulinum toxin type A is administered about one month prior to the onset of the season.

14. The method of claim 8, wherein the Botulinum toxin type A, is administered about two weeks to about six weeks prior to the onset of the season.

15. The method of claim 8, further comprising administering to the subject a therapeutically effective amount of an additional modality of treatment.

16. The method of claim 15, wherein the additional modality of treatment comprises a therapeutically effective amount of an anti-depressant.

17. The method of claim 16, wherein the anti-depressant is a selective serotonin reuptake inhibitor.

18. The method of claim 15, wherein the additional modality of treatment comprises light therapy.

19. A method for treating or preventing seasonal affective disorder in a subject, comprising administering a therapeutically effective amount of a Botulinum toxin A to a glabella of the subject to cause paralysis of a facial muscle in the glabella, thereby affecting the ability of the subject to scowl or appear sad, and thereby treating or preventing seasonal affective disorder in the subject, wherein the seasonal affective disorder is caused by onset of a specific season, and wherein the Botulinum toxin A is administered about two to about six weeks prior to the onset of the specific season.

20. The method of claim 19, wherein the specific season is winter.

21. The method of claim 19, wherein about 30 to about 50 Unit equivalents of Botulinum toxin type A is administered to the facial muscle.

22. The method of claim 21, further comprising administering an additional dose of about 30 to about 50 Unit equivalents of Botulinum toxin type A to the facial muscle after about two to six months.

23. The method of claim 21, comprising administering Botulinum toxin A to a corrugator supercilli and a procerus muscle of the subject.

24. The method of claim 19, further comprising administering to the subject a therapeutically effective amount of an additional modality of treatment.

25. The method of claim 24, wherein the additional modality of treatment is an antidepressant, psychotherapy, or light therapy.
